(19) United States
(12) Patent Application Publication
(10) Pub. No.: US 2025/0143588 A1
(43) Pub. Date: May 8, 2025

(54) SYSTEMS AND METHODS FOR MONITORING AND PREDICTING HEALTH OF A USER IN A FACILITY

(71) Applicant: HONEYWELL INTERNATIONAL INC., Charlotte, NC (US)

(72) Inventors: Hardik Agarwal, Siliguri (IN); Rajni Keshri, Bangalore (IN); Vineet Agrawal, Bangalore (IN)

(21) Appl. No.: 18/387,478

(22) Filed: Nov. 7, 2023

(51) Int. Cl. A61B 5/0205; A61B 5/00; A61B 5/11

(52) U.S. Cl. CPC A61B 5/0205; A61B 5/002; A61B 5/1113; A61B 5/7203; A61B 5/7253; A61B 5/7267; A61B 5/7282; A61B 5/746; A61B 5/747

(57) ABSTRACT

Various embodiments described herein relate to systems and methods for monitoring health of a user in a facility. In this regard, telemetry data from one or more sensors in a facility is received such that at least a portion of the telemetry data includes health data of a user in the facility. The health data is then filtered to determine heart rate signal and respiratory rate signal. The heart rate signal and respiratory rate signal is monitored over a pre-defined time period. The monitored heart rate signal and respiratory rate signal is then compared with one or more pre-defined thresholds. Based on the comparison of the monitored heart rate signal and respiratory rate signal, a possible health issue for the user is predicted by a machine learning algorithm. Further, in this regard, one or more alerts are generated based on the possible health issue that is predicted for the user.

### TECHNICAL FIELD

The present disclosure is related to monitoring health of a user/person in a facility. More particularly, the present disclosure relates to usage of contactless means for continuous monitoring and predicting health of the user in the facility.

### BACKGROUND

Monitoring health of a person is vital to assess and determine health issues associated with the person. Early detection of health issues helps in undertaking corrective actions and preventive measures by the person to take care of their health. Though we have numerous advanced medical solutions/devices available in the market, some of them are feasible only for commercial purposes (say for a hospital) and cannot be scaled at a domestic level for an individual user. For example, devices used in hospitals for monitoring health rate and breathing rate are generally high-priced and cannot be scaled at home for use of an individual. In another example, at times users cannot frequently commute to hospitals in order to get their health checked up as the users may be disabled, bedridden, or infants. In addition, the devices are complicated and not compact enough in order to be installed in an environment where there are space constraints. Accordingly, scaling down to monitor health of individuals amidst these constraints becomes challenging.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 illustrates a schematic diagram showing an exemplary environment comprising multiple facilities, in accordance with one or more example embodiments described herein.

FIG. 2 illustrates a schematic diagram showing an exemplary implementation of a controller that may execute techniques in accordance with one or more example embodiments described herein.

FIG. 3 illustrates a schematic diagram showing an exemplary health monitoring system in accordance with one or more example embodiments described herein.

FIG. 4 illustrates a schematic diagram showing an exemplary sensor assembly of a health monitoring system in a facility in accordance with one or more example embodiments described herein.

FIG. 5A illustrates a schematic diagram showing an exemplary data processing component of a health monitoring system in a facility in accordance with one or more example embodiments described herein.

FIG. 5B illustrates a schematic diagram showing an exemplary waveform representation of one or more resultant signals generated by a health monitoring system.

FIG. 6 illustrates a schematic diagram showing an exemplary implementation of a health monitoring system in a facility in accordance with one or more example embodiments described herein.

FIG. 7A illustrates a flowchart showing a method described in accordance with some example embodiments described herein.

FIG. 7B illustrates a flowchart showing a method described in accordance with some example embodiments described herein.

FIG. 8 illustrates a flowchart showing a method described in accordance with some example embodiments described herein.

FIG. 9 illustrates a flowchart showing a method described in accordance with some example embodiments described herein.

FIG. 10 illustrates a flowchart showing a method described in accordance with some example embodiments described herein.

FIG. 11 illustrates a flowchart showing a method described in accordance with some example embodiments described herein.

FIG. 12 illustrates a flowchart showing a method described in accordance with some example embodiments described herein.

FIG. 13 illustrates a flowchart showing a method described in accordance with some example embodiments described herein.

### SUMMARY

The details of some embodiments of the subject matter described in this specification are set forth in the accompanying drawings and the description below. Other features, aspects, and advantages of the subject matter will become apparent from the description, the drawings, and the claims.

In accordance with an example embodiment, a method is described. In one or more example embodiments, the method comprises receiving telemetry data from one or more sensors in a facility, wherein at least a portion of the telemetry data comprises health data of a user in the facility. In one or more example embodiments, the method comprises filtering the health data of the user to determine heart rate signal and respiratory rate signal. In one or more example embodiments, the method comprises monitoring the heart rate signal and the respiratory rate signal over a pre-defined time period. In one or more example embodiments, the method comprises comparing the monitored heart rate signal and respiratory rate signal with one or more pre-defined thresholds. In one or more example embodiments, the method comprises predicting by a machine learning algorithm, a possible health issue for the user based on the comparison of the monitored heart rate signal and respiratory rate signal. Further, in one or more example embodiments, the method comprises generating one or more alerts in response to predicting the possible health issue for the user.

The above summary is provided merely for purposes of providing an overview of one or more exemplary embodiments described herein so as to provide a basic understanding of some aspects of the disclosure. Accordingly, it will be appreciated that the above-described embodiments are merely examples and should not be construed to narrow the scope or spirit of the disclosure in any way. It will be appreciated that the scope of the disclosure encompasses many potential embodiments in addition to those here summarized, some of which are further explained in the following description and its accompanying drawings.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### DETAILED DESCRIPTION OF THE DRAWINGS

Reference will now be made in detail to embodiments, examples of which are illustrated in the accompanying drawings. In the following detailed description, numerous specific details are set forth in order to provide a thorough understanding of the various described example embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments. The term “or” is used herein in both the alternative and conjunctive sense, unless otherwise indicated. The terms “illustrative,” “example,” and “exemplary” are used to be examples with no indication of quality level. Like numbers refer to like elements throughout.

The phrases “in an embodiment,” “in one embodiment,” “according to one embodiment,” and the like generally mean that the particular feature, structure, or characteristic following the phrase can be included in at least one example embodiment of the present disclosure, and can be included in more than one example embodiment of the present disclosure (importantly, such phrases do not necessarily refer to the same example embodiment).

The word “exemplary” is used herein to mean “serving as an example, instance, or illustration.” Any implementation described herein as “exemplary” is not necessarily to be construed as preferred or advantageous over other implementations. If the specification states a component or feature “can,” “may,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature can be optionally included in some example embodiments, or it can be excluded.

One or more example embodiments of the present disclosure may provide an “Internet-of-Things” or “IoT” platform in a facility that uses real-time accurate models and visual analytics to monitor health of users. In addition, the platform provides predictions related to health of the users that are actionable and in turn result in sustained peak performance of the facility or the enterprise. The IoT platform is an extensible platform that is portable for deployment in any cloud or data center environment for providing an enterprise-wide, top to bottom view, displaying status of processes, assets, people, and/or safety. Further, the IoT platform of the present disclosure supports end-to-end capability to execute digital twins against process data to provide appropriate predictions related to health of the users.

An overall performance of a facility (e.g., a building, an industrial site, a factory, a warehouse, cockpit of an aerial vehicle, and/or the like) is directly or indirectly linked with performance of a user/personnel in the facility. Health condition of the user plays an important role on the performance of the user in the facility. Accordingly, it is vital to monitor health of the user in the facility in order to assess a health condition of the user. Though there are numerous advanced solutions available in the market, some of them are infeasible for use in the facility as they are not compact enough to be placed in the facility. For example, a machine used in a hospital for checking up health of a user may be incompatible in a cockpit of a fighter jet. Further, though several medical solutions exist in the market to monitor health of the user, some of them may not be viable at a domestic level. For example, a bulky machine used in a hospital may not be scaled down to a domestic level for an individual for example, in an environment like a house. Further, if the user in such environment is disabled, bedridden, infant, or unable to move, then monitoring health of such users becomes a challenging task.

Even though there are several wearable/portable solutions available in the market for health monitoring, there are several drawbacks associated with these solutions. Firstly, at times, wearable solutions do not provide a user with accurate readings and are prone to providing false or inaccurate readings. This may lead to false predictions regarding health of the user. Secondly, in some environments, usage of wearable solutions is prohibited. Some of the wearable solutions like smart watches have high electromagnetic fields and generally cause interference in those environments where there are radio magnetic fields. Further, the wearable solutions are always in physical contact when worn by the user causing discomfort. Provided that some of these solutions have high electromagnetic fields, the user wearing these solutions is always subjected to radiations which is not desirable. This may eventually affect health of the wearer. In addition, it is not safe for infants and/or aged people to use devices with high electromagnetic fields. Also, as these wearable devices have the capability of transferring data, there can be significant privacy related concerns as well. Thirdly, some of the portable solutions use imaging techniques to assess health condition of the user. As these imaging techniques capture images of the user, there can be significant privacy issues here as well. These constraints/drawbacks make health monitoring of the user a challenging task.

Thus, to address one or more constraints/drawbacks as observed above, various examples of systems and methods described herein relates to continuous health monitoring of a user using contactless means. Further, various example embodiments described herein also relates to providing predictions related to the health of the user based on the continuous health monitoring. In this regard, the user can undertake preventive measures to take care of one's health. Further, various examples of systems and methods described herein to monitor health of the user develop a solution that is compact, installable in a confined space, and versatile enough to be used across different domains. For example, the solution used in a hospital can be installed in a cockpit of an aerial vehicle. On the other hand, the solution can be used for an individual in an environment like house. Further, the exemplary systems and methods herein utilize one or more sensors that operate in that frequency which does not affect health of the user and does not have high electromagnetic fields. By this, signal interferences can be prevented. Given that various examples of systems and methods described herein are directed towards contactless health monitoring, there is no problem of discomfort due to continuous physical contact for the user. Also, the exemplary embodiments described herein utilize telemetry data from sensors to analyze vitals such as heart rate and respiratory rate of the user in order to monitor health of the user. The vitals monitored herein facilitate accurate assessments and predictions related to health of the user. Also, there is no requirement of capturing images of the user thereby eliminating privacy concerns completely.

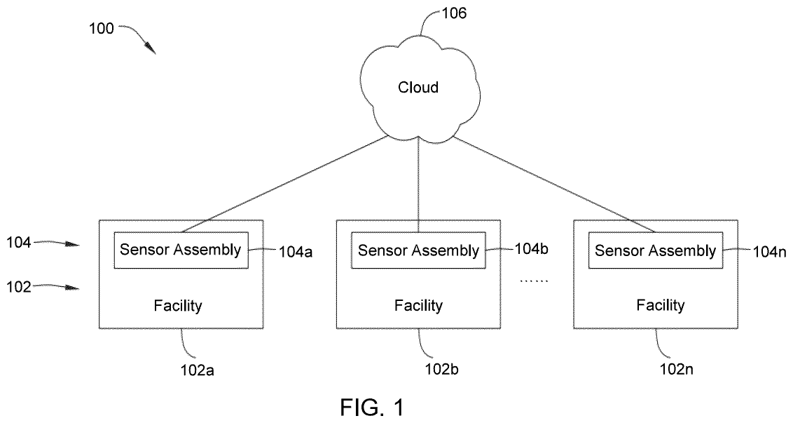

FIG. 1 illustrates a schematic diagram showing an exemplary environment comprising multiple facilities. According to various example embodiments described herein, an exemplary environment 100 comprises one or more facilities 102a, 102b, . . . 102n (collectively “facilities 102”). In some example embodiments, a facility of the one or more facilities 102a, 102b, . . . 102n may correspond to, for example, a residential complex, a commercial building, an institutional building, a monument, an IT park, a corporate office, an airport premises, a tourist place, a cockpit of an aerial vehicle, a hospital, a warehouse, a distribution center, a confined space, and/or the like. In some example embodiments, the one or more facilities 102a, 102b, . . . 102n in the illustrative environment 100 may be of same type. In some example embodiments, the one or more facilities 102a, 102b, . . . 102n in the illustrative environment 100 may be of different type. As it may be understood, these facilities may include one or more users/personnel who may be involved in performing various duties/tasks assigned to them. According to some example embodiments, these duties/tasks may require a user, at times, to move around the facility and/or be seated or stand idle at a place. In some example embodiments, at times, at least one user in a facility may be in continuous motion. For example, if a facility corresponds to a warehouse and personnel is performing a pick-and-place operation, then the personnel is in motion (say, walking on the floor) at times. Whereas in some other example embodiments, at times, at least one user in a facility may be seated or not in motion. For example, a pilot seated in a fighter jet is mostly static with no/less motion. In another example, a person who is seated or standing idle in one place in a facility is also mostly static with no/less motion. At times, it so happens that the user may not focus on health while performing a task and health issues go unobserved. Irrespective of whether the user is in motion or idle, it is vital to continuously monitor health of the user.

Accordingly, in one or more example embodiments described herein, each of the one or more facilities 102a, 102b, . . . 102n includes a respective sensor assembly 104a, 104b, . . . 104n (collectively “sensor assemblies 104”) to continuously monitor health of the user in each of the one or more facilities 102a, 102b, . . . 102n. In some example embodiments, each of one or more sensor assemblies 104a, 104b, . . . 104n may be placed in at least one portion of a respective facility. In some example embodiments, each of the one or more facilities 102a, 102b, . . . 102n may comprise more than one sensor assembly. In some example embodiments, each of the sensor assemblies 104 comprises one or more sensors. In some example embodiments, one or more sensors may correspond to Impulse Radio Ultrawide Band (IR-UWB) sensors and Passive Infrared (PIR) sensors. In some example embodiments, each of the one or more sensor assemblies 104a, 104b, . . . 104n is configured to continuously monitor user's health in the facility. In some example embodiments, one or more sensors in each of the one or more sensor assemblies 104a, 104b, . . . 104n are configured to continuously sense and receive telemetry data associated with the user's health. In some example embodiments, telemetry data can include time stamps and data values corresponding to those time stamps. In other words, at least a portion of the telemetry data may represent health data collected for a user over a period of time (e.g. continuous data stream captured over a time period). In this regard, in some example embodiments, the telemetry data sensed by the one or more sensors can correspond to one or more vitals that is to be monitored in order to assess user's health condition. In some example embodiments, one or more vitals corresponds to heart rate and respiratory rate.

Further, in some example embodiments, the one or more facilities 102a, 102b, . . . 102n can be operably coupled with a cloud 106, meaning that a communication between the cloud 106 and one or more facilities 102a, 102b, . . . 102n can be enabled. The cloud 106 may represent distributed computing resources, software, platform or infrastructure services which can enable data handling, data processing, data management, and/or analytical operations on the data exchanged & transacted amongst the various assets of the facilities 102. In this regard, in accordance with some example embodiments, telemetry data (e.g. sensor data) and optionally associated metadata (e.g. contextual information associated with sensor data) can be uploaded to the cloud 106 for processing. In this regard, in accordance with some example embodiments, cloud 106 can assess a health condition of the user. Further, in some example embodiments, the cloud 106 can also determine one or more health issues for the user and provide appropriate predictions related to the one or more health issues. Also, in some example embodiments, the cloud 106 can generate one or more alerts based at least in part on the predictions related to the one or more health issues.

In some example embodiments, the one or more sensor assemblies 104a, 104b, . . . 104n may operate as intermediary node to transact data between a respective facility and/or the cloud 106. In some example embodiments, each of the one or more sensor assemblies 104a, 104b, . . . 104n is capable of filtering telemetry data received from one or more sensors so as to be compatible with the cloud 106. In some example embodiments, each of the one or more facilities 102a, 102b, . . . 102n may comprise a respective gateway to transact data between a respective facility and/or the cloud 106. Accordingly, in some example embodiments, gateway may operate as intermediary node to transact data between a respective facility and/or the cloud 106. In an example embodiment, the cloud 106 includes one or more servers that may be programmed to communicate with the one or more facilities 102a, 102b, . . . 102n and to exchange data as appropriate. The cloud 106 may be a single computer server or may include a plurality of computer servers. In some example embodiments, the cloud 106 may represent a hierarchal arrangement of two or more computer servers, where perhaps a lower level computer server (or servers) processes telemetry data, for example, while a higher-level computer server oversees operation of the lower level computer server or servers.

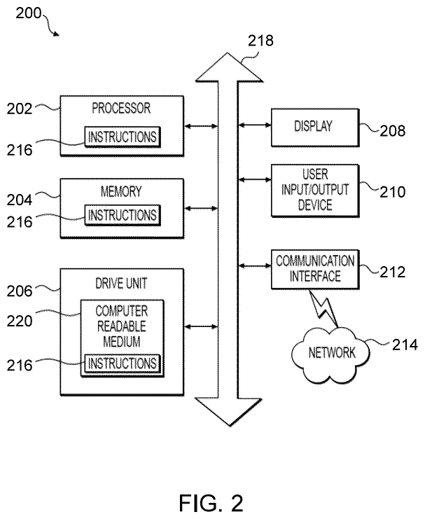

FIG. 2 illustrates a schematic diagram showing an exemplary implementation of a controller that may execute techniques in accordance with one or more example embodiments described herein. In one or more example embodiments, controller 200 described herein may include a set of instructions that can be executed to cause the controller 200 to perform any one or more of the methods or computer based functions disclosed herein. The controller 200 may operate as a standalone device or may be connected, e.g., using a network, to other computer systems or peripheral devices.

In a networked deployment, the controller 200 may operate in the capacity of a server or as a client in a server-client user network environment, or as a peer computer system in a peer-to-peer (or distributed) network environment. The controller 200 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the controller 200 can be implemented using electronic devices that provide voice, video, or data communication. Further, while the controller 200 is illustrated as a single system, the term “system” shall also be taken to include any collection of systems or sub-systems that individually or jointly execute a set, or multiple sets, of instructions to perform one or more computer functions.

As illustrated in FIG. 2, the controller 200 may include a processor 202, e.g., a central processing unit (CPU), a graphics processing unit (GPU), or both. The processor 202 may be a component in a variety of systems. For example, the processor 202 may be part of a standard computer. The processor 202 may be one or more general processors, digital signal processors, application specific integrated circuits, field programmable gate arrays, servers, networks, digital circuits, analog circuits, combinations thereof, or other now known or later developed devices for analyzing and processing data. The processor 202 may implement a software program, such as code generated manually (i.e., programmed).

The controller 200 may include a memory 204 that can communicate via a bus 218. The memory 204 may be a main memory, a static memory, or a dynamic memory. The memory 204 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 204 includes a cache or random-access memory for the processor 202. In alternative implementations, the memory 204 is separate from the processor 202, such as a cache memory of a processor, the system memory, or other memory. The memory 204 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 204 is operable to store instructions executable by the processor 202. The functions, acts or tasks illustrated in the figures or described herein may be performed by the processor 202 executing the instructions stored in the memory 204. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the controller 200 may further include a display 208, such as a liquid crystal display (LCD), an organic light emitting diode (OLED), a flat panel display, a solid-state display, a cathode ray tube (CRT), a projector, a printer or other now known or later developed display device for outputting determined information. The display 208 may act as an interface for the user to see the functioning of the processor 202, or specifically as an interface with the software stored in the memory 204 or in the drive unit 206. Additionally or alternatively, the controller 200 may include an input/output device 210 configured to allow a user to interact with any of the components of controller 200. The input/output device 210 may be a number pad, a keyboard, or a cursor control device, such as a mouse, or a joystick, touch screen display, remote control, or any other device operative to interact with the controller 200. The controller 200 may also or alternatively include drive unit 206 implemented as a disk or optical drive. The drive unit 206 may include a computer-readable medium 220 in which one or more sets of instructions 216, e.g. software, can be embedded. Further, the instructions 216 may embody one or more of the methods or logic as described herein. The instructions 216 may reside completely or partially within the memory 204 and/or within the processor 202 during execution by the controller 200. The memory 204 and the processor 202 also may include computer-readable media as discussed above.

In some systems, a computer-readable medium 220 includes instructions 216 or receives and executes instructions 216 responsive to a propagated signal so that a device connected to a network 214 can communicate voice, video, audio, images, or any other data over the network 214. Further, the instructions 216 may be transmitted or received over the network 214 via a communication port or interface 212, and/or using a bus 218. The communication port or interface 212 may be a part of the processor 202 or may be a separate component. The communication port or interface 212 may be created in software or may be a physical connection in hardware. The communication port or interface 212 may be configured to connect with a network 214, external media, the display 208, or any other components in controller 200, or combinations thereof. The connection with the network 214 may be a physical connection, such as a wired Ethernet connection or may be established wirelessly as discussed below. Likewise, the additional connections with other components of the controller 200 may be physical connections or may be established wirelessly. The network 214 may alternatively be directly connected to a bus 218.

While the computer-readable medium 220 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 220 may be non-transitory, and may be tangible. The computer-readable medium 220 can include a solid-state memory such as a memory card or other package that houses one or more non-volatile read-only memories. The computer-readable medium 220 can be a random-access memory or other volatile re-writable memory. Additionally or alternatively, the computer-readable medium 220 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The controller 200 may be connected to a network 214. The network 214 may define one or more networks including wired or wireless networks. The wireless network may be a cellular telephone network, an 802.11, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 214 may include wide area networks (WAN), such as the Internet, local area networks (LAN), campus area networks, metropolitan area networks, a direct connection such as through a Universal Serial Bus (USB) port, or any other networks that may allow for data communication. The network 214 may be configured to couple one computing device to another computing device to enable communication of data between the devices. The network 214 may generally be enabled to employ any form of machine-readable media for communicating information from one device to another. The network 214 may include communication methods by which information may travel between computing devices. The network 214 may be divided into sub-networks. The sub-networks may allow access to all of the other components connected thereto or the sub-networks may restrict access between the components. The network 214 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof. It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

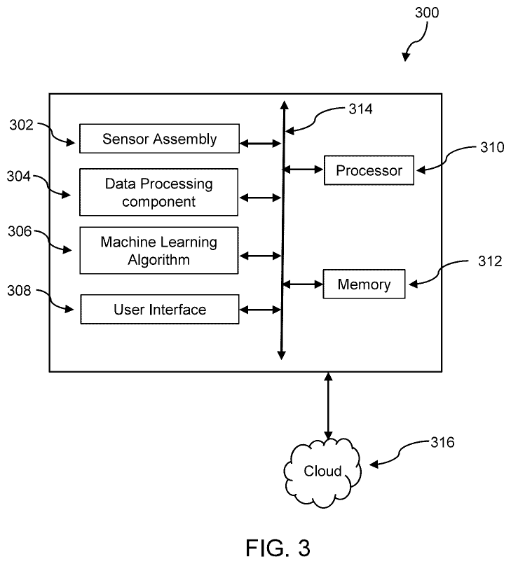

FIG. 3 illustrates a schematic diagram showing an exemplary health monitoring system in accordance with one or more example embodiments described herein. In one or more example embodiments, health monitoring system 300 described herein continuously monitors health of a user in a facility. In this regard, in one or more example embodiments described herein, the health monitoring system 300 is configured to receive data associated with the user's health in the facility. In one or more example embodiments, the health monitoring system 300 analyzes data to assess health condition of the user in the facility. In this regard, in some exemplary embodiments, the system 300 may facilitate a practical application of identification of one or more health issues by pattern recognition in the data. Also, in some example embodiments, the health monitoring system 300 can construct a model associated with the user's health based on the received data. Further, in one or more example embodiments, the health monitoring system 300 can use the model to provide one or more predictions based on the health condition of the user. Accordingly, in some example embodiments, the system 300 facilitates a practical application of data analytics technology and/or digital transformation technology to continuously monitor health of the desired user in facility.

In an example embodiment, the health monitoring system 300 is a server system (e.g., a server device) that facilitates a data analytics platform between one or more computing devices, one or more data sources, and/or one or more assets. In one or more example embodiments, the health monitoring system 300 is a device with one or more processors and a memory. Also, in some example embodiments, the health monitoring system 300 is implementable via the cloud 316. The health monitoring system 300 is implementable in one or more facilities related to one or more technologies, for example, but not limited to, enterprise technologies, connected building technologies, industrial technologies, Internet of Things (IoT) technologies, data analytics technologies, digital transformation technologies, cloud computing technologies, cloud database technologies, server technologies, network technologies, private enterprise network technologies, wireless communication technologies, machine learning technologies, artificial intelligence technologies, digital processing technologies, electronic device technologies, computer technologies, supply chain analytics technologies, aircraft technologies, industrial technologies, cybersecurity technologies, navigation technologies, asset visualization technologies, oil and gas technologies, petrochemical technologies, refinery technologies, process plant technologies, procurement technologies, and/or one or more other technologies.

In some example embodiments, the health monitoring system 300 comprises one or more components such as, a sensor assembly 302, a data processing component 304, a machine learning algorithm 306, and/or a user interface 308. Additionally, in one or more example embodiments, the health monitoring system 300 comprises a processor 310 and/or a memory 312. In one or more example embodiments, one or more components of the health monitoring system 300 may be communicatively coupled to processor 310 and/or a memory 312 via a bus 314. In certain example embodiments, one or more aspects of the health monitoring system 300 (and/or other systems, apparatuses and/or processes disclosed herein) constitute executable instructions embodied within a computer-readable storage medium (e.g., the memory 312). For instance, in an example embodiment, the memory 312 stores computer executable component and/or executable instructions (e.g., program instructions). Furthermore, the processor 310 facilitates execution of the computer executable components and/or the executable instructions (e.g., the program instructions). In an example embodiment, the processor 310 is configured to execute instructions stored in memory 312 or otherwise accessible to the processor 310.

The processor 310 is a hardware entity (e.g., physically embodied in circuitry) capable of performing operations according to one or more embodiments of the disclosure. Alternatively, in an example embodiment where the processor 310 is embodied as an executor of software instructions, the software instructions configure the processor 310 to perform one or more algorithms and/or operations described herein in response to the software instructions being executed. In an example embodiment, the processor 310 is a single core processor, a multi-core processor, multiple processors internal to the health monitoring system 300, a remote processor (e.g., a processor implemented on a server), and/or a virtual machine. In certain example embodiments, the processor 310 is in communication with the memory 312, the sensor assembly 302, the data processing component 304, the machine learning algorithm 306, and/or the user interface 308 via the bus 314 to, for example, facilitate transmission of data between the processor 310, the memory 312, the sensor assembly 302, the data processing component 304, the machine learning algorithm 306, and/or the user interface 308. In some example embodiments, the processor 310 may be embodied in a number of different ways and, in certain example embodiments, includes one or more processing devices configured to perform independently. Additionally or alternatively, in one or more example embodiments, the processor 310 includes one or more processors configured in tandem via bus 314 to enable independent execution of instructions, pipelining of data, and/or multi-thread execution of instructions.

The memory 312 is non-transitory and includes, for example, one or more volatile memories and/or one or more non-volatile memories. In other words, in one or more example embodiments, the memory 312 is an electronic storage device (e.g., a computer-readable storage medium). The memory 312 is configured to store information, data, content, one or more applications, one or more instructions, or the like, to enable the health monitoring system 300 to carry out various functions in accordance with one or more embodiments disclosed herein. In accordance with some example embodiments described herein, the memory 312 may correspond to an internal or external memory of the health monitoring system 300. In some examples, the memory 312 may correspond to a database communicatively coupled to the health monitoring system 300. As used herein in this disclosure, the term “component,” “system,” and the like, is a computer-related entity. For instance, “a component,” “a system,” and the like disclosed herein is either hardware, software, or a combination of hardware and software. As an example, a component is, but is not limited to, a process executed on a processor, a processor circuitry, an executable component, a thread of instructions, a program, and/or a computer entity.

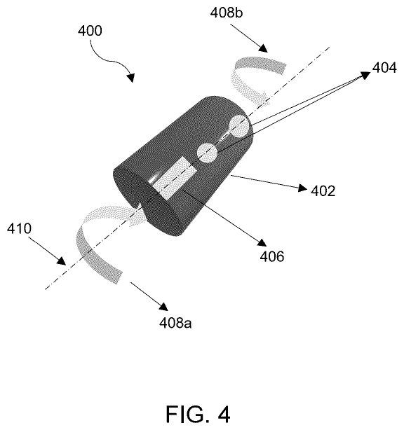

In one or more example embodiments, the sensor assembly 302 may be installed in at least a portion of the facility where the user's health is to be monitored. In one or more example embodiments, the sensor assembly 302 comprises one or more sensors configured to continuously monitor the user's health. In this regard, the one or more sensors continuously sense data that is required to monitor the user's health. In some example embodiments, this data may correspond to telemetry data such that at least a portion of the telemetry data comprises health data of the user. In some example embodiments, the health data comprises one or more vitals that is required to monitor the user's health. In some example embodiments, the one or more vitals may correspond to heart rate and respiratory rate of the user. An exemplary sensor assembly is described in more details in accordance with FIG. 4 of the current disclosure.

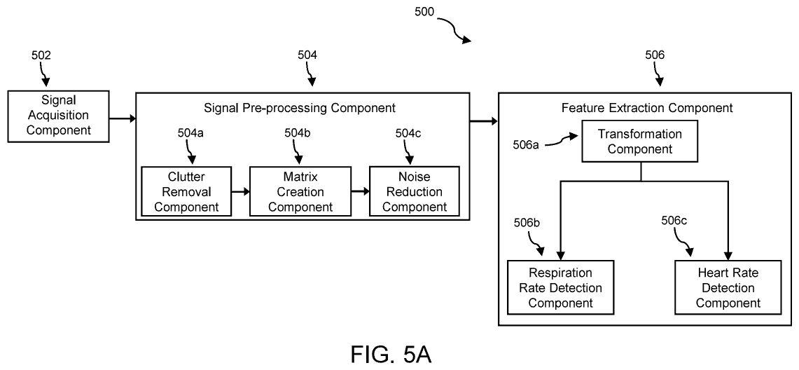

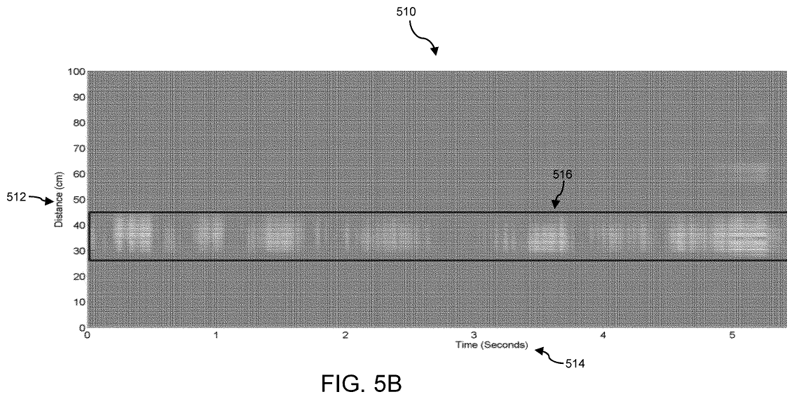

In some example embodiments, the data processing component 304 is configured to analyze the telemetry data received from the sensor assembly 302. In some example embodiments, the data processing component 304 is configured to detect health data from the telemetry data. Further, in some example embodiments, the data processing component 304 extracts one or more vitals from the health data based on the analysis of health data. In this regard, in some example embodiments, the data processing component 304 is configured to apply a filter to health data in order to extract heart rate and respiratory rate of the user. Also, in some example embodiments, the data processing component 304 is configured to separate the extracted heart rate and respiratory rate. Further, in some example embodiments, the data processing component 304 is configured to monitor signals corresponding to the heart rate and respiratory rate, respectively for a pre-defined time period. In this regard, in some example embodiments, the data processing component 304 is configured to define one or more patterns for the heart rate signal and the respiratory rate signal for the pre-defined time period. Further, in some example embodiments, the data processing component 304 is configured to determine a position of the user based at least in part on analysis of the telemetry data. An exemplary data processing component is also described in more details in accordance with FIG. 5A of the current disclosure.

Further, in some example embodiments, the machine learning algorithm 306 of the health monitoring system 300 is configured to provide one or more predictions related to user's health. In this regard, the machine learning algorithm 306 provides the one or more predictions based at least in part on the one or more patterns for the heart rate signal and the respiratory rate signal. In some exemplary embodiments, the one or more predictions corresponds to one or more health issues associated with the user's health. In some example embodiments, the one or more predictions corresponds to a possibility of one or more health issues associated with the user's health. In some example embodiments, the one or more health issues can correspond to heart attack or respiratory problems. In some example embodiments, the one or more predictions corresponds to a remedial measure or a precautionary measure that is to be taken by the user. In some example embodiments, the machine learning algorithm 306 is trained with datasets in order to provide the one or more predictions. In some example embodiments, the datasets may correspond to electrocardiogram (ECG) data. Further, in some example embodiments, ECG data can be associated with people of one or more age groups. Also, in some example embodiments, ECG data can be associated with people suffering from one or more health issues associated with heart and respiratory system. Further, in some example embodiments, the machine learning algorithm 306 is configured to define one or more pre-defined thresholds. In some example embodiments, the one or more pre-defined thresholds defined are based at least in part on ECG data associated with people of one or more age groups and people suffering from one or more health issues associated with heart and respiratory system. In some example embodiments, the machine learning algorithm 306 compares the one or more patterns for the heart rate signal and the respiratory rate signal with the one or more pre-defined thresholds. Further, in some example embodiments, the machine learning algorithm 306 provides the one or more predictions if the one or more patterns for the heart rate signal and the respiratory rate signal does not meet the one or more pre-defined thresholds. In some example embodiments, the one or more predictions can be provided as a feedback to the machine learning algorithm 306. In this regard, the machine learning algorithm 306 learns over time to provide improved and accurate predictions. Also, in some example embodiments, the machine learning algorithm 306 can be trained with one or more new datasets on a regular basis or for a pre-defined time interval.

Further, in some example embodiments, the one or more predictions provided by the machine learning algorithm 306 can be transmitted to the user interface 308. In some example embodiments, the user interface 308 can correspond to an interface of a device associated with the user in the facility. In some example embodiments, the user interface 308 can correspond to an interface of a device associated with a supervisor of the user in the facility. In some example embodiments, the user interface 308 can correspond to an interface of a device associated with a family member of the user in the facility. In some example embodiments, the user interface 308 can correspond to an interface of a device associated with a rescue/emergency team in the facility. In some example embodiments, the user interface 308 can correspond to an interface of a device associated with a medical personnel in the facility. In some example embodiments, one or more alert signals can be generated based on the one or more predictions provided by the machine learning algorithm 306. In some example embodiments, the one or more alert signals can be transmitted to the user interface 308. In this regard, in some example embodiments, one or more notifications can be generated on the user interface 308 based on the one or more alert signals. Accordingly, in some example embodiments, the one or more notifications can be visual notifications. Whereas, in some example embodiments, the one or more notifications can be audio notifications. Further, in some example embodiments described herein, the one or more predictions can be used to undertake one or more actions in order to take care of user's health. For example, an action can correspond to a suggestion for taking a medical test. In another example, an action can correspond to a suggestion for taking a break/rest. In another example, an action can correspond to transmission of the heart rate signal, the respiratory rate signal, and/or other health vitals by the health monitoring system 300 to an appropriate focal. For example, a focal may correspond to a medical personnel or emergency team. In another example, a focal may correspond to a supervisor of the user.

In some example embodiments, one or more components, processor 310 and/or a memory 312 of the health monitoring system 300 may be communicatively coupled to the cloud 316 over a network. In this regard, the one or more components, processor 310 and/or a memory 312 along with the cloud 316 can continuously monitor health of the user in a facility. In some example embodiments, the network may be for example, a Wi-Fi network, a Near Field Communications (NFC) network, a Worldwide Interoperability for Microwave Access (WiMAX) network, a personal area network (PAN), a short-range wireless network (e.g., a Bluetooth® network), an infrared wireless (e.g., IrDA) network, an ultra-wideband (UWB) network, an induction wireless transmission network, a BACnet network, a NIAGARA network, a NIAGARA CLOUD network, and/or another type of network. In some example embodiments, telemetry data received from the sensor assembly 302 can be transmitted to the cloud 316. According to some example embodiments, the health monitoring system 300 may additionally comprise a gateway. In this regard, the gateway may be configured to transmit telemetry data received from the sensor assembly 302 to the cloud 316. Further, in some example embodiments, the cloud 316 can be configured to perform analysis on the telemetry data, extract heart rate and respiratory rate, and/or separate the extracted heart rate and respiratory rate. In some example embodiments, the cloud 316 may transmit the extracted heart rate and respiratory rate back to the data processing component 304 for further processing. For example, if the health monitoring system 300 is used to monitor health of a pilot in an aerial vehicle (say, a fighter jet), then telemetry data received from the sensor assembly 302 can be transmitted to a ground station via flight management system (FMS) of the aerial vehicle. In this example embodiment, the FMS can be configured to perform one or more operations performed by the data processing component 304 in order to monitor health of the pilot. Accordingly, in this example embodiment, FMS acts as cloud 316. In some example embodiments, the cloud 316 may be configured to predict one or more health issues for the user. In this regard, the cloud 316 may transmit one or more notifications to the user interface 308 based on the prediction. Also, in some example embodiments, the cloud 316 may be configured to create a model from the health data of the user. In some example embodiments, the cloud 316 may be configured to perform one or more operations/functionalities of one or more components, processor 310 and/or a memory 312 of the health monitoring system 300 in order to monitor health of the user in the facility.

FIG. 4 illustrates a schematic diagram showing an exemplary sensor assembly of a health monitoring system in a facility in accordance with one or more example embodiments described herein. In some example embodiments, exemplary sensor assembly 400 described herein corresponds to one or more sensor assemblies 104a, 104b, . . . 104n described in FIG. 1 of the current disclosure. In some example embodiments, the sensor assembly 400 is installed in at least a portion of a facility where a user's health is to be monitored. According to various example embodiments described herein, the sensor assembly 400 comprises one or more sensors 404 and 406 placed in an enclosure 402. Further, in some example embodiments, the one or more sensors 404 and 406 can correspond to Impulse Radio Ultrawide Band (IR-UWB) sensor and Passive Infrared (PIR) sensor, respectively. In some example embodiments described herein, a range or a field of view of operation of the one or more sensors 404 and 406 may be pre-defined. In this regard, in some example embodiments, the range or the field of view of operation may be defined based at least in part on a type of facility and a nature of task performed by the user in the facility. In some example embodiments, the sensor assembly 400 also comprises a processor placed in the enclosure 402. According to some example embodiments, the processor may be similar to processor 202 or processor 310. Further, in some example embodiments, the sensor assembly 400 comprises a rotatory motor (not shown) placed in the enclosure 402. Also, in some example embodiments, the sensor assembly 400 also comprises one or more gears which are coupled to the rotatory motor. The one or more gears described in some example embodiments herein translate rotational motion into linear movement so that the rotatory motor moves in a desired direction. In some example embodiments, the one or more sensors 404 and 406 may be communicatively coupled with the rotatory motor. Further, in some example embodiments, the enclosure 402 of sensor assembly 400 also comprises a power source to supply power to the one or more sensors 404 and 406, processor, rotatory motor, and/or one or more gears. In some example embodiments, the power source may be a battery. Further, in some example embodiments, the enclosure 402 may be one of: a plastic project box, a 3D printed enclosure, an electronics enclosure, or a modular enclosure. In some example embodiments, the enclosure 402 may comprise one or more compartments/sections. In some example embodiments, the one or more compartments/sections may be designed based at least in part on a size of the one or more sensors 404 and 406, processor, rotatory motor, one or more gears, and/or power source. Further, in some example embodiments, the one or more compartments/sections may be designed based at least in part on an accessibility and/or ventilation for the one or more sensors 404 and 406, processor, rotatory motor, one or more gears, and/or power source. In this regard, in some example embodiments, the one or more compartments/sections may facilitate secure placement of the one or more sensors 404 and 406, processor, rotatory motor, one or more gears, and/or power source in the sensor assembly 400. Accordingly, in one or more example embodiments, the sensor assembly 400 described herein can be used to monitor health of the user irrespective of whether the user is moving or idle within the facility.

In one or more example embodiments, IR-UWB sensor 404 of the sensor assembly 400 is configured to transmit one or more signals towards the user in the facility. In this regard, in one or more example embodiments, a transmitter of IR-UWB sensor 404 transmits the one or more signals towards the user. Accordingly, in some example embodiments, the one or more signals may penetrate the user's body and reflect back from one or more internal organs/structure of the user's body. For example, the one or more signals may reflect back from heart and/or lungs of the user's body. Further, in one or more example embodiments, the IR-UWB sensor 404 is configured to receive one or more reflected signals back from the user. In this regard, in one or more example embodiments, a receiver of IR-UWB sensor 404 receives the one or more reflected signals. In some example embodiments, the one or more reflected signals comprise one or more vitals that are required to monitor user's health. In some example embodiments, the one or more reflected signals also comprise information related to one or more movements within user's body. In this regard, in some example embodiments, the one or more vitals can be heart rate and respiratory rate. In some example embodiments, one or more signals that are transmitted and received using the IR-UWB sensor 404 can be used to determine a location of the user in the facility. In some example embodiments, the processor of the sensor assembly 400 determines the location of the user in the facility. In this regard, in one example embodiment, the processor of the sensor assembly 400 identifies a time of flight (ToF), two way ranging (TWR), a time difference of arrival (TDoA), and/or an angle of arrival (AoA) of the one or more signals that are transmitted and received using the IR-UWB sensor 404 to determine the location of the user in the facility. Further, in another example embodiment, the processor of the sensor assembly 400 creates a map/layout of the facility that includes a location of the IR-UWB sensor 404 in the facility and/or an identifier or label of the IR-UWB sensor 404. Accordingly, in some example embodiments, the processor identifies based on analysis of the one or more signals that are transmitted and received using the IR-UWB sensor 404, the location of the IR-UWB sensor 404 and/or the identifier or label of the IR-UWB sensor 404 to determine the location of the user in the facility.

Further, in some exemplary embodiments, the PIR sensor 406 of the sensor assembly 400 is configured to detect motion of the user within a pre-defined range or a pre-defined field of view in the facility. In this regard, the PIR sensor 406 measures infrared radiation levels radiated from the user in its field of view. In some example embodiments, the PIR sensor 406 can measure infrared radiation levels for predefined time period to detect motion of the user within the facility. Also, in some example embodiments, the PIR sensor 406 compares measured infrared radiation levels with a predefined threshold to detect motion of the user within the facility. In response to a determination that the infrared radiation levels exceed the predefined threshold, in some example embodiments, the PIR sensor 406 determines motion of the user. Accordingly, in one or more example embodiments, the PIR sensor 406 is configured to continuously track, in its field of view, motion of the user in the facility. With this, in one or more example embodiments, the PIR sensor 406 is further configured to continuously track a position of the user in the facility. In some example embodiments, based on the continuous tracking of the user's position, a signal is transmitted from the PIR sensor 406 to the rotatory motor. In this regard, in some example embodiments, in response to receiving the signal, the rotatory motor moves the sensor assembly 400 based on the position of the user. Also, in some example embodiments described herein, the signal transmitted from the PIR sensor 406 also comprises at least one of: a torque, a current, an angle, and a speed required to operate the rotatory motor and/or one or more gears. In some example embodiments, at least one of: a torque, a current, an angle, and a speed is determined based at least in part on the position of the user. Accordingly, in some example embodiments, the sensor assembly 400 is moved appropriately to ensure that the user is within the field of view of the one or more sensors 404 and 406. In some example embodiments, the rotatory motor rotates the sensor assembly 400 based on the position of the user to have the user within the field of view of the one or more sensors 404 and 406. Said alternatively, the sensor assembly 400 focuses or is directed along a direction of the user's position whether the user is in motion or idle. In some example embodiments, the rotatory motor is configured to move the sensor assembly 400 along axis 410. In some example embodiments, the rotatory motor can pivot the sensor assembly 400 upwards along the axis 410. In some example embodiments, the rotatory motor can pivot the sensor assembly 400 downwards along the axis 410. In some example embodiments described herein, the one or more gears coupled to the rotatory motor facilitate the motion of the rotatory motor upwards or downwards. Also, in some example embodiments, the rotatory motor is configured to rotate the sensor assembly 400 along axis 410. In some example embodiments, the sensor assembly 400 can be rotated in a clockwise direction 408a along the axis 410. In some example embodiments, the sensor assembly 400 can be rotated in an anti-clockwise direction 408b along the axis 410. With this, in one or more example embodiments, the sensor assembly 400 can be adjusted based on the user's position by the rotatory motor to capture the one or more vitals for continuous health monitoring. In this regard, in one or more example embodiments described herein, the IR-UWB sensor 404 can continuously sense the one or more reflected signals that are required to monitor user's health irrespective of whether the user is in motion or idle in the facility.

Further, in some example embodiments, the processor of the sensor assembly 400 tracks a position of the sensor assembly 400 to determine if the user is within the field of view. In this regard, in some example embodiments, the processor constantly tracks movement of the rotatory motor, movement of one or more gears, torque and current supplied to the rotatory motor, angular position of the rotatory motor, and/or speed of the rotatory motor to determine a position of the sensor assembly 400. In some example embodiments, the processor utilizes movement of the rotatory motor, movement of one or more gears, torque and current supplied to the rotatory motor, angular position of the rotatory motor, and/or speed of the rotatory motor to determine if the sensor assembly 400 needs to be re-positioned. In some example embodiments, processor of the sensor assembly 400 transmits one or more signals to operate the rotatory motor and/or one or more gears to move the sensor assembly 400 to an appropriate position along the axis 410. With this, in some example embodiments, the sensor assembly 400 can be posited along specific angles and/or positions to have the user within the field of view of the one or more sensors 404 and 406. Also, in some example embodiments, a position in which the sensor assembly 400 is to be posited (say, a first position) may be pre-defined. Further, in some example embodiments, a time for which the sensor assembly 400 is to be posited in the first position may be pre-defined. Accordingly, the processor in some example embodiments may transmit a signal to the rotatory motor and/or one or more gears to reposition the sensor assembly 400 to a rest position or another desired position (say, a second position) based at least in part on the pre-defined position and/or the pre-defined time.

FIG. 5A illustrates a schematic diagram showing an exemplary data processing component of a health monitoring system in a facility in accordance with one or more example embodiments described herein. In some example embodiments, exemplary data processing component 500 described herein corresponds to data processing component 304 described in FIG. 3 of the current disclosure. In this regard, data processing component 500 can comprise a signal acquisition component 502, a signal pre-processing component 504, and/or a feature extraction component 506. Initially, in some example embodiments, the signal acquisition component 502 receives one or more raw signals. In some example embodiments, the one or more raw signals can be received from sensor assembly 400. In some example embodiments, the one or more raw signals can correspond to health data. Further, in some example embodiments, the one or more raw signals can correspond to one or more reflected signals received at IR-UWB sensor 404 of the sensor assembly 400. In this regard, in some example embodiments, the one or more raw signals can comprise one or more signals reflected back from one or more internal organs/structure of the user's body (for example, heart and/or lungs). Further, in some example embodiments, the one or more raw signals can also comprise information related to one or more movements within user's body. Accordingly, in some example embodiments, the one or more raw signals can comprise information related to one or more vitals that is required to monitor user's health. In this regard, in some example embodiments, the one or more vitals correspond to heart rate and respiratory rate. Also, in some example embodiments, the one or more raw signals can additionally comprise one or more noise signals and/or clutter signals.

Further, in some example embodiments, the signal acquisition component 502 can transmit the one or more raw signals to the signal pre-processing component 504. In some example embodiments, the signal pre-processing component 504 processes the one or more raw signals. In this regard, in some example embodiments, the signal pre-processing component 504 comprises a clutter removal component 504a, a matrix creation component 504b, and/or a noise reduction component 504c. In some example embodiments, the clutter removal component 504a processes the one or more raw signals to remove the one or more clutter signals. In some example embodiments, the one or more clutter signals comprise one or more unwanted/interference signals. In this regard, according to some example embodiments, the clutter removal component 504a uses one or more filters to remove the one or more clutter signals. For example, a filter of the one or more filters may be a loopback filter. Upon removal of the one or more clutter signals, in some example embodiments, the clutter removal component 504a generates one or more resultant signals. An exemplary representation of one or more resultant signals is described in more details in accordance with FIG. 5B of the current disclosure. Further, in some example embodiments, the one or more resultant signals can be transmitted to the matrix creation component 504b. According to some example embodiments, the matrix creation component 504b further processes the one or more resultant signals. In this regard, in some example embodiments, the matrix creation component 504b processes the one or more resultant signals to determine a location of one or more internal organs/structure of the user's body. For example, the matrix creation component 504b can determine a location of chest in the user's body. Further, in some example embodiments the matrix creation component 504b determines one or more raw waveforms of one or more vitals upon determination of location of one or more internal organs/structure of the user's body. For example, the one or more vitals can be heart rate and respiratory rate.

Upon determination of one or more raw waveforms of one or more vitals, in some example embodiments, the matrix creation component 504b combines the one or more raw waveforms to create a matrix. In this regard, according to some example embodiments, a size of the matrix can be “m”×“n” (m cross n). In some example embodiments, “m” represents a length of a coarse time scale and “n” represents a length of a fine time scale for each of the one or more raw waveforms. The matrix creation component 504b can select a value of “m” in some example embodiments. According to this aspect, in some example embodiments, the matrix creation component 504b selects the value of “m” to have a balance between frequency resolution and rate of change in each of the one or more raw waveforms. For example, the value of “m” is selected to have a balance between frequency resolution and rate of change in each waveforms for heart rate and respiratory rate. In some example embodiments, the frequency resolution indicates a level with which the matrix creation component 504b can detect values corresponding to the one or more vitals in the one or more raw waveforms. Said alternatively, in some example embodiments, an accuracy with which the matrix creation component 504b can detect values corresponding to the one or more vitals is represented by the frequency resolution. In some example embodiments, a higher value of “m” results in a slower change in the one or more vitals whereas a lower value of “m” results in a faster change in the one or more vitals. Said alternatively, in some example embodiments, a higher value of “m” facilitates detection of values corresponding to the one or more vitals in the one or more raw waveforms even at lower frequencies. In some example embodiments, at least one value in the matrix of size “m”×“n” can be related with a periodic motion caused by contraction and/or relaxation cycles of internal organs (for example, lungs and heart) of user's body.

Further, in some example embodiments, the matrix creation component 504b can identify a column of interest from the matrix of size “m”×“n”. In some example embodiments, a column of one or more columns of the matrix that exhibits a highest variance along the fine time scale is identified as the column of interest. In this regard, in some example embodiments, one or more values in the column of interest represent that position of the user which has maximum movements and that are apt to monitor user's health. Also, in some example embodiments, identification of the column of interest makes sure that only relevant information is considered to monitor user's health. Accordingly, this eliminates noise and/or redundant values that are not required to monitor user's health. Further, in some example embodiments, the matrix creation component 504b processes the one or more values in the column of interest to extract one or more vital signals for one or more vitals of the user's body. Upon extraction of one or more vital signals, in some example embodiments, the matrix creation component 504b can transmit the one or more vital signals to noise reduction component 504c. In some example embodiments, the noise reduction component 504c can apply a filter to the one or more vital signals to further eliminate high-frequency noise and/or baseline drift. In this regard, the filter applied by the noise reduction component 504c can be a Kalman filter. In some example embodiments, the noise reduction component 504c generates one or more filtered vital signals upon applying the filter.

In some example embodiments, the one or more filtered vital signals comprise multiple signals of one or more vitals. Said alternatively, the one or more filtered vital signals can have a combination of signals of one or more vitals. To analyze the health of the user and to individually assess each vital, in some example embodiments, one or more filtered vital signals are segregated and extracted. For example, with heart rate and respiratory rate as the one or more vitals, one or more filtered vital signals can comprise signals corresponding to both heart rate and respiratory rate. Accordingly, in some example embodiments, the noise reduction component 504c transmits the one or more filtered vital signals to the feature extraction component 506. To analyze heart rate and respiratory rate individually, the feature extraction component 506 can identify characteristic components of the one or more vitals in the one or more filtered vital signals in some example embodiments. For example, the feature extraction component 506 can determine peaks and/or periodic variations in the one or more filtered vital signals to identify the one or more vitals. Further, in some example embodiments, the feature extraction component 506 comprises a transformation component 506a, a respiration rate detection component 506b, and/or a heart rate detection component 506c. In some example embodiments, initially the one or more filtered vital signals are in time domain. At the transformation component 506a, in some example embodiments, the one or more filtered vital signals in time domain are transformed into frequency domain. In this regard, the transformation component 506a utilizes Fourier transform to transform the one or more filtered vital signals from time domain to frequency domain. The transformation component 506a generates one or more transformed vital signals upon application of transformations on the one or more filtered vital signals.

Further, in some example embodiments, the one or more transformed vital signals is transmitted to the respiration rate detection component 506b. In some example embodiments, the respiration rate detection component 506b detects periodic variations for breathing in the one or more transformed vital signals to identify respiratory rate. Also, in some embodiments, the respiration rate detection component 506b performs frequency analysis on the one or more transformed vital signals to identify respiratory rate. Accordingly, in some example embodiments, the respiration rate detection component 506b isolates one or more respiratory signals corresponding to respiratory rate from the one or more transformed vital signals. In some example embodiments, the heart rate detection component 506c detects for one or more high peaks in the one or more transformed vital signals within a pre-defined range. For example, the pre-defined range may be 10-30 cycles per minute. In some example embodiments, a location of the one or more high peaks is detected in the one or more transformed vital signals. In some example embodiments, the one or more high peaks are further classified to be the respiratory rate. Further, in some example embodiments, the one or more high peaks in integer multiples of the pre-defined range are also classified to be the respiratory rate. Accordingly, the heart rate detection component 506c isolates one or more respiratory signals corresponding to the respiratory rate. Also, in some example embodiments, the heart rate detection component 506c tracks a number of the one or more high peaks in the one or more transformed vital signals. Further, in some example embodiments, the heart rate detection component 506c detects one or more peaks with highest frequencies from amongst the one or more high peaks. Accordingly, in some example embodiments, the heart rate detection component 506c identifies a location of the one or more peaks. In some example embodiments, the heart rate detection component 506c considers one or more values at the location of the one or more peaks to determine heart rate. In this regard, in some example embodiments, the heart rate detection component 506c determines an average of the one or more values to determine the heart rate. Further, in some example embodiments, the respiratory rate and the heart rate can be stored for example, in the memory 312.

FIG. 5B illustrates a schematic diagram showing an exemplary waveform representation of one or more resultant signals generated by a health monitoring system. In some example embodiments described herein, exemplary waveform representation of one or more resultant signals 510 can be generated by clutter removal component 504a of data processing component 304. According to some example embodiments, the clutter removal component 504a generates one or more resultant signals 516. In some example embodiments, the waveform representation of one or more resultant signals 510 comprises axes 512 and 514. Further, in some example embodiments, axis 512 represents a distance between user's body and sensor assembly 400. In this regard, in some example embodiments, the distance between user's body and sensor assembly 400 can be related to periodic motion caused by contraction and/or relaxation cycles of internal organs (for example, lungs and heart) in user's body. Whereas, in some example embodiments, axis 514 represents time. In this regard, in some example embodiments, the one or more resultant signals 516 comprise values corresponding to one or more vitals which can be used by matrix creation component 504b to create a matrix.

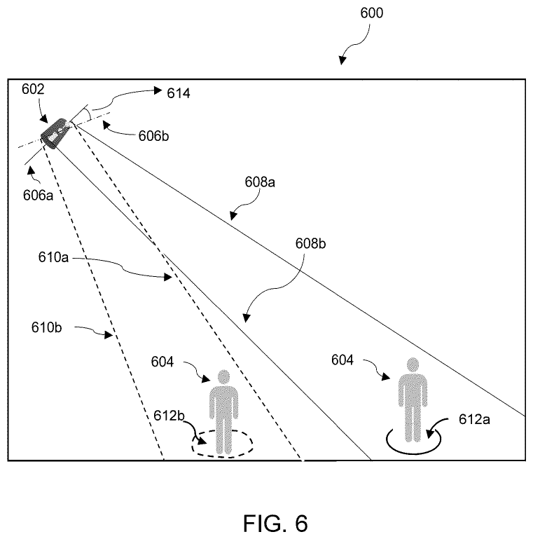

FIG. 6 illustrates a schematic diagram showing an exemplary implementation of a health monitoring system in a facility in accordance with one or more example embodiments described herein. According to some example embodiments, exemplary environment 600 may correspond to at least a portion of the facility where health of a user 604 is to be monitored. In some example embodiments, the facility may be one of one or more facilities 102a, 102b, . . . 102n as described in FIG. 1 of the current disclosure. Further, in some example embodiments, an exemplary sensor assembly 602 is used to monitor health of the user 604 in the facility 600. According to some example embodiments, the sensor assembly 602 described herein can correspond to sensor assembly 400 described in FIG. 4 of the current disclosure. Accordingly, in some example embodiments, the sensor assembly 602 comprises one or more sensors such as Impulse Radio Ultrawide Band (IR-UWB) sensor and Passive Infrared (PIR) sensor, and a rotatory motor.

In some exemplary embodiments, the sensor assembly 602 is installed in the environment 600 to monitor health of the user 604. For example, the environment 600 may correspond to a warehouse where the user 604 is performing a pick-and-place operation of one or more packages in the warehouse. In this example, the sensor assembly 602 may be installed in a portion of the warehouse where the user 604 is performing the pick-and-place operation. Initially, in some example embodiments, the user 604 can be initially positioned at 612a (say, a first position) in the environment 600. For example, at position 612a, the user 604 may be standing idle as the user 604 may be awaiting one or more instructions corresponding to a task (say, a pick operation). In some example embodiments, when the user 604 is positioned at 612a, the sensor assembly 602 is posited along axis 606a. At this point, in some example embodiments, the sensor assembly 602 can have a first field of view so that the user 604 at position 612a is within the first field of view. According to some example embodiments, the first field of view is defined between lines 608a and 608b. In some example embodiments, the sensor assembly 602 along the axis 606a continuously receives telemetry data via one or more sensors for monitoring health of the user 604 at position 612a.

Further, in some example embodiments, the user 604 positioned at 612a may receive an instruction to perform the task. In an example embodiment, the instruction may require the user 604 to move to a position 612b (say, a second position) in order to perform the task. Accordingly, the user 604 moves to position 612b from position 612a. In some example embodiments, one or more sensors of the sensor assembly 602 determine that the user 604 has moved to position 612b. In this regard, the PIR sensor of the sensor assembly 602 determines that the user 604 has moved to position 612b. In some example embodiments, the rotatory motor moves the sensor assembly 602 from axis 606a to axis 606b upon the determination that the user 604 has moved to position 612b from 612a. In some example embodiments, when the sensor assembly 602 is along the axis 606b, the sensor assembly 602 can have a second field of view so that the user 604 at position 612b is also within the second field of view. In some example embodiments, the second field of view is defined between lines 610a and 610b. Accordingly, in some example embodiments, the rotatory motor pivots the sensor assembly 602 along the axis 606a by an angle 614 in order to move the sensor assembly 602 from axis 606a to axis 606b. Also, in some example embodiments, the rotatory motor may also rotate the sensor assembly 602 in clockwise direction. In some example embodiments, the sensor assembly 602 along the axis 606b continuously receives telemetry data via one or more sensors for monitoring health of the user 604 at position 612b. In this regard, in one or more example embodiments, the sensor assembly 602 continuously tracks position of the user 604 in the environment 600 to continuously receive sense data that is required to monitor the health of the user 604.

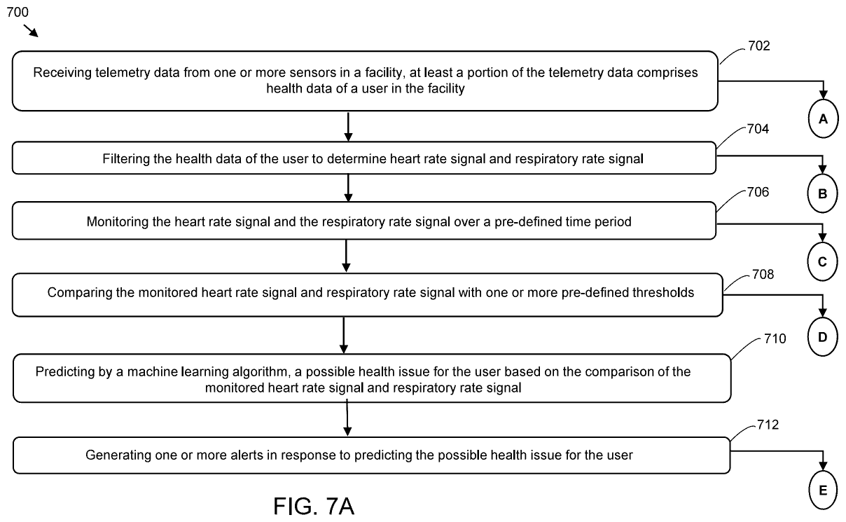

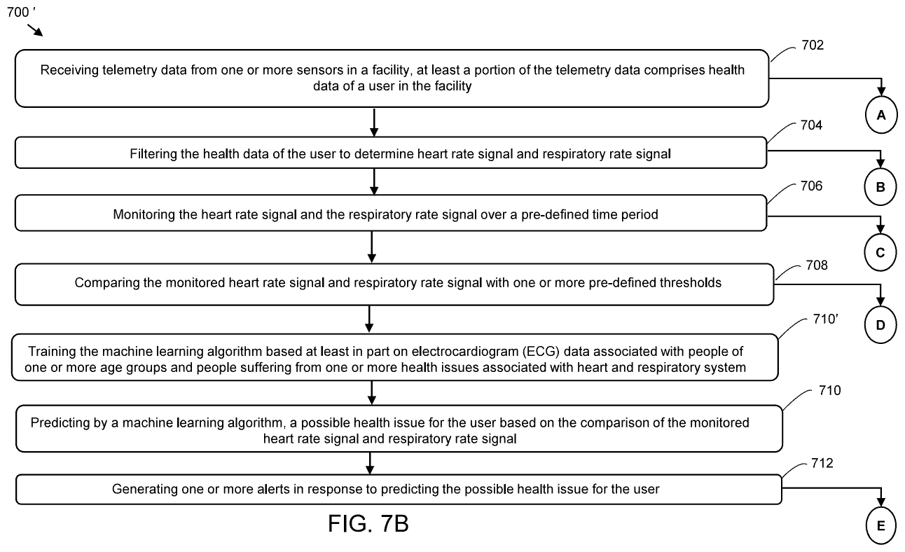

FIG. 7A illustrates a flowchart showing a method described in accordance with some example embodiments described herein. An exemplary flowchart 700 describes an exemplary method for monitoring health of a user in a facility in accordance with some example embodiments described herein. At step 702, health monitoring system 300 includes means, such as sensor assembly 302 to receive telemetry data from one or more sensors in the facility. In this regard, at least a portion of the telemetry data comprises health data of the user in the facility.

At step 704, health monitoring system 300 includes means, such as data processing component 304 to filter the health data of the user to determine heart rate signal and respiratory rate signal. At step 706, health monitoring system 300 includes means, such as data processing component 304 to monitor the heart rate signal and the respiratory rate signal over a pre-defined time period. At step 708, health monitoring system 300 includes means, such as machine learning algorithm 306 to compare the monitored heart rate signal and respiratory rate signal with one or more pre-defined thresholds. At step 710, health monitoring system 300 includes means, such as machine learning algorithm 306 to predict a possible health issue for the user based on the comparison of the monitored heart rate signal and respiratory rate signal. At step 712, health monitoring system 300 includes means, such as machine learning algorithm 306 to generate one or more alerts in response to predicting the possible health issue for the user.

FIG. 7B illustrates a flowchart showing a method described in accordance with some example embodiments described herein. An exemplary flowchart 700′ describes another exemplary method for monitoring health of a user in a facility in accordance with some example embodiments described herein. In some example embodiments, in addition to the steps described in FIG. 7A of the current disclosure, FIG. 7B can comprise an additional step 710′. At step 710′, health monitoring system 300 includes means, such as machine learning algorithm 306 that is trained based at least in part on electrocardiogram (ECG) data associated with people of one or more age groups and people suffering from one or more health issues associated with heart and respiratory system.

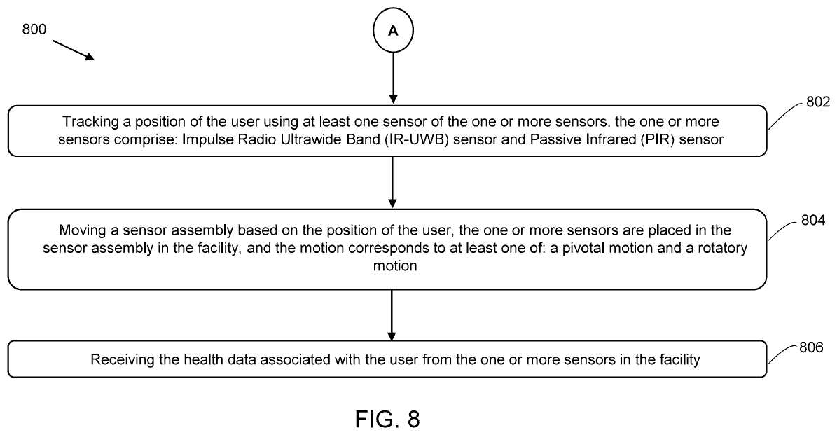

FIG. 8 illustrates a flowchart showing a method described in accordance with some example embodiments described herein. An exemplary flowchart 800 describes an exemplary method for receiving telemetry data from one or more sensors in a facility in accordance with some example embodiments described herein. At step 802, health monitoring system 300 includes means, such as sensor assembly 302 to track a position of a user using at least one sensor of the one or more sensors. In this regard, in some example embodiments, the one or more sensors comprise: Impulse Radio Ultrawide Band (IR-UWB) sensor and Passive Infrared (PIR) sensor.

At step 804, health monitoring system 300 includes means, such as sensor assembly 302 to move the sensor assembly based on the position of the user. In this regard, in some example embodiments, the one or more sensors are placed in the sensor assembly in the facility. Also, in some example embodiments, the motion corresponds to at least one of: a pivotal motion and a rotatory motion. At step 806, health monitoring system 300 includes means, such as sensor assembly 302 to receive health data associated with the user from the one or more sensors in the facility.

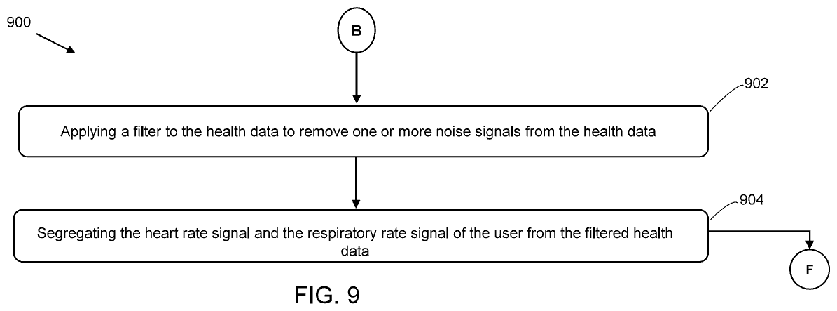

FIG. 9 illustrates a flowchart showing a method described in accordance with some example embodiments described herein. An exemplary flowchart 900 describes an exemplary method for filtering health data of a user in a facility. At step 902, health monitoring system 300 includes means, such as data processing component 304 to apply a filter to the health data to remove one or more noise signals from the health data. At step 904, health monitoring system 300 includes means, such as data processing component 304 to segregate heart rate signal and respiratory rate signal of the user from the filtered health data.

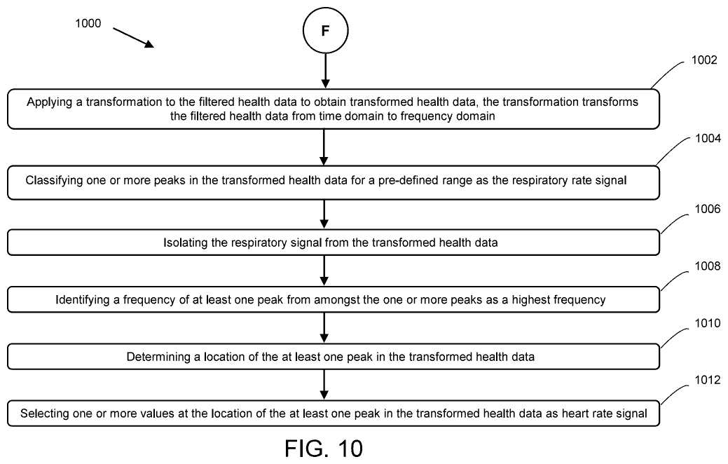

FIG. 10 illustrates a flowchart showing a method described in accordance with some example embodiments described herein. An exemplary flowchart 1000 describes an exemplary method for segregating heart rate signal and respiratory rate signal of a user. At step 1002, health monitoring system 300 includes means, such as data processing component 304 to apply a transformation to filtered health data to obtain transformed health data. In some example embodiments, the transformation transforms the filtered health data from time domain to frequency domain. At step 1004, health monitoring system 300 includes means, such as data processing component 304 to classify one or more peaks in the transformed health data for a pre-defined range as the respiratory rate signal. At step 1006, health monitoring system 300 includes means, such as data processing component 304 to isolate the respiratory signal from the transformed health data. At step 1008, health monitoring system 300 includes means, such as data processing component 304 to identify a frequency of at least one peak from amongst the one or more peaks as a highest frequency. At step 1010, health monitoring system 300 includes means, such as data processing component 304 to determine a location of the at least one peak in the transformed health data. At step 1012, health monitoring system 300 includes means, such as data processing component 304 to select one or more values at the location of the at least one peak in the transformed health data as heart rate signal.

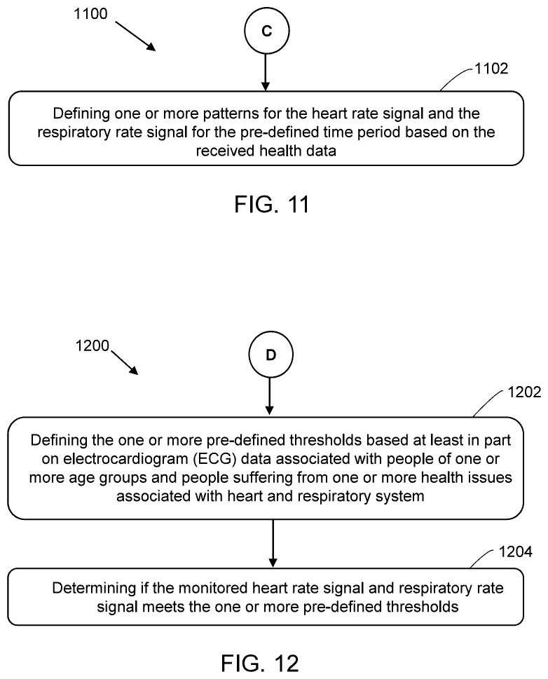

FIG. 11 illustrates a flowchart showing a method described in accordance with some example embodiments described herein. An exemplary flowchart 1100 describes an exemplary method for monitoring heart rate signal and respiratory rate signal of a user. At step 1102, health monitoring system 300 includes means, such as data processing component 304 to define one or more patterns for the heart rate signal and the respiratory rate signal for a pre-defined time period based on received health data.

FIG. 12 illustrates a flowchart showing a method described in accordance with some example embodiments described herein. An exemplary flowchart 1200 describes an exemplary method for comparing monitored heart rate signal and respiratory rate signal with one or more pre-defined thresholds. At step 1202, health monitoring system 300 includes means, such as machine learning algorithm 306 to define the one or more pre-defined thresholds based at least in part on electrocardiogram (ECG) data associated with people of one or more age groups and people suffering from one or more health issues associated with heart and respiratory system. At step 1204, health monitoring system 300 includes means, such as machine learning algorithm 306 to determine if the monitored heart rate signal and respiratory rate signal meets the one or more pre-defined thresholds.

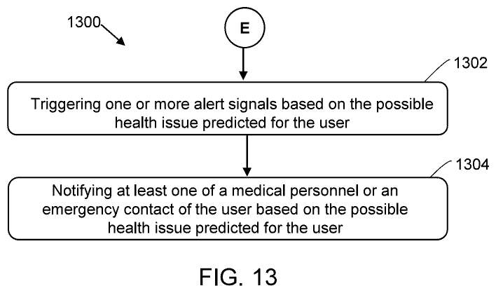

FIG. 13 illustrates a flowchart showing a method described in accordance with some example embodiments described herein. An exemplary flowchart 1300 describes an exemplary method for generating one or more alerts. At step 1302, health monitoring system 300 includes means, such as machine learning algorithm 306 to trigger one or more alert signals based on a possible health issue predicted for a user. At step 1304, health monitoring system 300 includes means, such as machine learning algorithm 306 and user interface 308 to notify at least one of a medical personnel or an emergency contact of the user based on the possible health issue predicted for the user.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the supply management system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

1. A method for monitoring health of a user in a facility, the method comprising:
receiving telemetry data from one or more sensors in the facility, wherein at least a portion of the telemetry data comprises health data of the user in the facility;
filtering the health data of the user to determine heart rate signal and respiratory rate signal;
monitoring the heart rate signal and the respiratory rate signal over a pre-defined time period;
comparing the monitored heart rate signal and respiratory rate signal with one or more pre-defined thresholds;
predicting by a machine learning algorithm, a possible health issue for the user based on the comparison of the monitored heart rate signal and respiratory rate signal; and
generating one or more alerts in response to predicting the possible health issue for the user.

2. The method of claim 1, wherein receiving telemetry data from the one or more sensors in the facility comprises:
tracking a position of the user using at least one sensor of the one or more sensors, wherein the one or more sensors comprise: Impulse Radio Ultrawide Band (IR-UWB) sensor and Passive Infrared (PIR) sensor;
moving a sensor assembly based on the position of the user, wherein the one or more sensors are placed in the sensor assembly in the facility, and wherein the motion corresponds to at least one of: a pivotal motion and a rotatory motion; and
receiving the health data associated with the user from the one or more sensors in the facility.

3. The method of claim 1, wherein filtering the health data of the user comprises:
applying a filter to the health data to remove one or more noise signals from the health data; and
segregating the heart rate signal and the respiratory rate signal of the user from the filtered health data.

4. The method of claim 3, wherein segregating the heart rate signal and the respiratory rate signal comprises:
applying a transformation to the filtered health data to obtain transformed health data, wherein the transformation transforms the filtered health data from time domain to frequency domain;
classifying one or more peaks in the transformed health data for a pre-defined range as the respiratory rate signal;
isolating the respiratory signal from the transformed health data;
identifying a frequency of at least one peak from amongst the one or more peaks as a highest frequency;
determining a location of the at least one peak in the transformed health data; and
selecting one or more values at the location of the at least one peak in the transformed health data as heart rate signal.

5. The method of claim 1, wherein monitoring the heart rate signal and the respiratory rate signal comprises:
defining one or more patterns for the heart rate signal and the respiratory rate signal for the pre-defined time period based on the received health data.

6. The method of claim 1, wherein comparing the monitored heart rate signal and respiratory rate signal with one or more pre-defined thresholds comprises:
defining the one or more pre-defined thresholds based at least in part on electrocardiogram (ECG) data associated with people of one or more age groups and people suffering from one or more health issues associated with heart and respiratory system; and
determining if the monitored heart rate signal and respiratory rate signal meets the one or more pre-defined thresholds.

7. The method of claim 1, further comprising:
training the machine learning algorithm based at least in part on electrocardiogram (ECG) data associated with people of one or more age groups and people suffering from one or more health issues associated with heart and respiratory system.

8. The method of claim 1, wherein generating one or more alerts comprises at least one of:
triggering one or more alert signals based on the possible health issue predicted for the user; and
notifying at least one of a medical personnel or an emergency contact of the user based on the possible health issue predicted for the user.

9. A system for monitoring health of a user in a facility, the system comprising:
a sensor assembly, wherein the sensor assembly comprises one or more sensors;
a processor;
a memory communicatively coupled to the processor, wherein the memory comprises one or more instructions which when executed by the processor cause the system to:
receive telemetry data from the one or more sensors, wherein at least a portion of the telemetry data comprises health data of the user in the facility;
filter the health data of the user to determine heart rate signal and respiratory rate signal;
monitor the heart rate signal and the respiratory rate signal over a pre-defined time period;
compare the monitored heart rate signal and respiratory rate signal with one or more pre-defined thresholds;
predict by a machine learning algorithm, a possible health issue for the user based on the comparison of the monitored heart rate signal and respiratory rate signal; and
generate one or more alerts in response to predicting the possible health issue for the user.

10. The system of claim 9, wherein the system is further configured to:
track a position of the user using at least one sensor of the one or more sensors, wherein the one or more sensors comprise: Impulse Radio Ultrawide Band (IR-UWB) sensor and Passive Infrared (PIR) sensor;
move the sensor assembly based on the position of the user, wherein the motion corresponds to at least one of: a pivotal motion and a rotatory motion; and
receive the health data associated with the user from the one or more sensors in the facility.

11. The system of claim 9, wherein the system is further configured to:
apply a filter to the health data to remove one or more noise signals from the health data; and
segregate the heart rate signal and the respiratory rate signal of the user from the filtered health data.

12. The system of claim 11, wherein to segregate the heart rate signal and the respiratory rate signal of the user, the system is further configured to:
apply a transformation to the filtered health data to obtain transformed health data, wherein the transformation transforms the filtered health data from time domain to frequency domain;
classify one or more peaks in the transformed health data for a pre-defined range as the respiratory rate signal;
isolate the respiratory signal from the transformed health data;
identify a frequency of at least one peak from amongst the one or more peaks as a highest frequency;
determine a location of the at least one peak in the transformed health data; and
select one or more values at the location of the at least one peak in the transformed health data as heart rate signal.

13. The system of claim 9, wherein the system is further configured to:
define one or more patterns for the heart rate signal and the respiratory rate signal for the pre-defined time period based on the received health data.

14. The system of claim 9, wherein the system is further configured to:
define the one or more pre-defined thresholds based at least in part on electrocardiogram (ECG) data associated with people of one or more age groups and people suffering from one or more health issues associated with heart and respiratory system; and
determine if the monitored heart rate signal and respiratory rate signal meets the one or more pre-defined thresholds.

15. The system of claim 9, wherein the system is further configured to:
train the machine learning algorithm based at least in part on electrocardiogram (ECG) data associated with people of one or more age groups and people suffering from one or more health issues associated with heart and respiratory system.

16. The system of claim 9, wherein the system is further configured to:
trigger one or more alert signals based on the possible health issue predicted for the user; and
notify at least one of a medical personnel or an emergency contact of the user based on the possible health issue predicted for the user.

17. A non-transitory, computer-readable storage medium having stored thereon executable instructions that, when executed by one or more processors, cause the one or more processors to:
receive telemetry data from one or more sensors, wherein at least a portion of the telemetry data comprises health data of a user in a facility;
filter the health data of the user to determine heart rate signal and respiratory rate signal;
monitor the heart rate signal and the respiratory rate signal over a pre-defined time period;
compare the monitored heart rate signal and respiratory rate signal with one or more pre-defined thresholds;
predict by a machine learning algorithm, a possible health issue for the user based on the comparison of the monitored heart rate signal and respiratory rate signal; and
generate one or more alerts in response to predicting the possible health issue for the user.

18. The non-transitory, computer-readable storage medium of claim 17, wherein the one or more processors is further configured to:
track a position of the user using at least one sensor of the one or more sensors, wherein the one or more sensors comprise: Impulse Radio Ultrawide Band (IR-UWB) sensor and Passive Infrared (PIR) sensor;
move a sensor assembly based on the position of the user, wherein the one or more sensors are placed in the sensor assembly in the facility, and wherein the motion corresponds to at least one of: a pivotal motion and a rotatory motion; and
receive the health data associated with the user from the one or more sensors in the facility.

19. The non-transitory, computer-readable storage medium of claim 17, wherein the one or more processors is further configured to:
apply a filter to the health data to remove one or more noise signals from the health data; and
segregate the heart rate signal and the respiratory rate signal of the user from the filtered health data.

20. The non-transitory, computer-readable storage medium of claim 17, wherein the one or more processors is further configured to:
define the one or more pre-defined thresholds based at least in part on electrocardiogram (ECG) data associated with people of one or more age groups and people suffering from one or more health issues associated with heart and respiratory system; and
determine if the monitored heart rate signal and respiratory rate signal meets the one or more pre-defined thresholds;
train the machine learning algorithm based at least in part on the ECG data associated with people of one or more age groups and people suffering from one or more health issues associated with heart and respiratory system;
trigger one or more alert signals based on the possible health issue predicted for the user; and
notify at least one of a medical personnel or an emergency contact of the user based on the possible health issue predicted for the user.
